(19) United States
(12) Patent Application Publication
(10) Pub. No.: US 2019/0112470 A1
(43) Pub. Date: Apr. 18, 2019

(54) COLORED OXYGEN SCAVENGING COMPOSITIONS REQUIRING NO INDUCTION PERIOD

(71) Applicant: GRAHAM PACKAGING COMPANY, L.P., Lancaster, PA (US)

(72) Inventors: Murali K. Akkapeddi, York, PA (US); Brian A. Lynch, Merrimack, NH (US)

(21) Appl. No.: 15/782,561

(22) Filed: Oct. 12, 2017

(51) Int. Cl. C08L 67/02; C08K 5/098; C08K 5/103; C08K 3/32; C08K 3/08; C08J 5/18; C08G 81/02; C08G 63/82; C07C 69/587

(52) U.S. Cl. CPC C08L 67/02; C08K 5/098; C08K 5/103; C08K 3/32; C08K 3/08; C08G 2650/04; C08G 81/027; C08G 81/025; C08G 63/82; C07C 69/587; C08J 5/18

(57) ABSTRACT

The present invention provides a composition comprising: a polyester base polymer; an oxidizable polyether-based additive; a transition metal catalyst; a colorant; and a polyunsaturated fatty ester additives, wherein the polyester base polymer is substantially free of antimony and substantially free of phosphorous. Containers made from the composition are colored and exhibit excellent oxygen scavenging properties with no induction period.

### FIELD OF THE INVENTION

The present invention relates to compositions useful for preparing containers that scavenge oxygen to protect oxygen sensitive contents. In particular, the present invention relates to compositions useful for preparing containers that scavenge oxygen without a delay in the onset of oxygen scavenging when a colorant additive is added and the colorant additive causes a delay in the onset of oxygen scavenging.

### BACKGROUND OF THE INVENTION

All references, including publications, patent applications, and patents, cited herein are hereby incorporated by reference to the same extent as if each reference were individually and specifically indicated to be incorporated by reference and were set forth in its entirety herein.

It is well known that oxygen-sensitive food products such as tomato-based ketchups and sauces, and beverage products such as orange juice, beer, and green tea, require a packaging material that has either high oxygen barrier properties or the ability to block any oxygen ingress so as to preserve the freshness and flavor of the packaged contents. Traditionally, metal and glass packaging (e.g., cans or jars) has been used as oxygen-impervious containers.

However, in recent years, plastic containers, and more particularly injection stretch blow molded polyethylene terephthalate (PET) containers have made significant inroads into packaging, replacing the metal and glass containers for at least reasons of better economics, lighter weight, increased breakage resistance, and better consumer acceptance. Such PET containers have enjoyed widespread use in packaging at least because the biaxial orientation of PET polymer chains leads to a unique combination of clarity, toughness and moderate gas barrier properties. However, there is still a need to enhance the oxygen barrier of PET containers even further in order to extend its use in the packaging of highly oxygen sensitive food and beverage products.

Although the containers may have a single layer of PET (i.e., monolayer containers) or be comprised of more than one layer of PET (i.e., multilayer containers), for cost reasons, monolayer PET containers are typically preferred over multilayer PET-containers because the multilayer process requires more expensive equipment and operational costs. While clear monolayer, oxygen scavenging PET containers satisfy a majority of consumer application needs such as the ketchup bottles & juice bottles, the food service industry, such as the restaurants, have additional need for highly colored bottles (e.g., red pigmented PET ketchup bottles or yellow-pigmented PET mustard bottles) mainly for aesthetic reasons. Such colored monolayer bottles must also exhibit sufficient oxygen scavenging properties for long shelf life.

For efficient oxygen scavenging to occur, a polymer material or additive used in packaging must chemically react immediately with the permeating oxygen so as to block the oxygen ingress into the container. Oxygen scavenging is typically thought to be a free radical initiated oxidation reaction between oxygen from air and an oxidizable polymer or additive used in the packaging. Oxygen scavenging compositions also typically employ suitable transition metal salts such as cobalt carboxylates as oxygen scavenging catalysts. Due to the free radical nature of oxygen scavenging process care must be taken to avoid free radical inhibiting impurities or additives in the PET resin used for such applications.

Use of certain polyamides in combination with a transition metal is known to be useful as an oxygen scavenging material. One particularly useful polyamide is PA-MXD6 which contains meta-xylene residues in the polymer chain. See, for example, U.S. Pat. Nos. 5,639,815; 5,049,624; and 5,021,515.

U.S. Pat. Nos. 6,083,585 and 6,558,762 to Cahill disclose oxygen scavenging polyester compositions wherein the oxygen scavenging component is polybutadiene-PET block copolymer and the catalyst for the oxygen scavenging material is transition metal salts.

U.S. Pat. No. 6,455,620 to Cyr et. al., discloses the use of polyethers selected from polyalkylene glycols, their copolymers, and blends thereof as oxygen scavengers in PET.

U.S. Patent Application Publications US2012/0114887 and US2012/0214935 disclose the use of copolyetheresters as oxygen scavengers in PET.

While the oxygen scavengers found in the references above find utility in packaging, there are still some drawbacks that include lengthy induction periods before oxygen-scavenging activity is achieved and or life spans (capacities) which may be too short. For example, molded containers that employ diamides such as, for example, dibenzyl adipamide (DBA) as the oxygen scavenger may have an induction period (i.e., a delay in the of up to three months at ambient temperature and humidity or up to four weeks at elevated temperature (38° C.) and humidity (85% RH) after the bottles are filled with deoxygenated water.

The introduction of a colorant can also significantly impact induction times. For example, molded containers that employ PET and a polyether based scavenger such as, for example, Oxyclear® 3500, may scavenge in certain containers without any induction period, however, the addition of a colorant such as, for example, a red or yellow dye, can interfere and cause an undesireable induction period of approximately 5 weeks or in more severe cases complete oxygen-scavenging inhibition.

Induction periods are not acceptable in real commercial practice where plastic containers are made and filled immediately (or shortly thereafter) with an oxygen-sensitive food or beverage product. The oxygen scavenging must occur immediately after filling to protect the taste and nutrient qualities of the food and/or beverage products contained within. In some instances, such deficiencies can be partially addressed by increasing the level of oxygen scavenger or the oxidation catalyst, but this invariably results in not only increased cost but also many undesirable effects such as haze, decreased melt viscosity, poor processability, and recyclability issues.

Thus, there is a need in the art for effective oxygen scavenging compositions that can be colored and still eliminate any induction period for oxygen scavenging such that prolonged aging or conditioning of formed containers is not needed.

### BRIEF SUMMARY OF THE INVENTION

Embodiments of the present invention include compositions including a substantially antimony-free and substantially phosphorous-free polyester base polymer; an oxidizable polyether-based additive; a transition metal catalyst, a colorant, and a polyunsaturated fatty ester additive

The polyester base polymer is preferably polyethylene terephthalate and in one embodiment preferably contains less than 100 ppm of antimony and phosphorous, more preferably less than 50 ppm of antimony and phosphorous, even more preferably less than 10 ppm of antimony and phosphorous, and most preferably contains between about 0 ppm and about 2 ppm of antimony and phosphorus. In another embodiment, the polyester base polymer preferably includes 46 ppm or less of antimony and phosphorus, more preferably 40 ppm or less of antimony and phosphorus, even more preferably 31.4 ppm or less of antimony and phosphorus, and most preferably 15.7 ppm or less of antimony and phosphorus.

The oxidizable polyether-based additive has the general formula X—[R—O]ₙ—R′—Y, where R is a substituted or unsubstituted alkylene chain having from 2 to 10 carbon atoms; n ranges from 4 to 100; X and Y are selected from the group consisting of: H, OH, —OCOR₁, —OCOAr₁, —OR₁ and —OAr₁; and R′ may be the same as R or selected from the group consisting of —[COR₂COOR₃O]ₚ— and —[COAr₂COOR₃O]ₚ—. R₁ is an alkyl group having from 2 to 18 carbon atoms, Ar₁ is an aryl group, Ar₂ is a phenylene or naphthylene group, R₂ and R₃ are C₂ to C₁₈ alkylene groups, and p ranges from 10 to 100. The oxidizable polyether-based additive is preferably selected from polyether diols, ester capped derivatives of polyether diols, polyether-polyester block copolymers, and ether-capped derivatives of polyetherdiols (e.g., α,ω-polyether diethers). Preferable among the polyether diols is polytetramethylene ether glycol, preferable among the ester capped derivatives of polyether diols are polytetramethylene ether glycol dibenzoate and polytetramethylene ether glycol dioctaoate, and preferable among the polyether-polyester block copolymers is PTMEG-b-PET copolymer. Preferable among the α,ω-polyether diethers are PTMEG-α,ω-dimethyl ether or PTMEG-α,ω-diethyl ether. The oxidizable polyether based additive may make up at least 0.5 wt. % of the composition, preferably about 1 wt. % to about 5 wt. %.

The transition metal catalyst preferably is a transition metal salt of cobalt. The counterion of the transition metal salt is preferably a carboxylate counterion. In a preferred embodiment, the transition metal salt is cobalt neodecanoate.

The polyunsaturated fatty ester additive preferably comprises of a linear or branched hydrocarbon moiety having two or more unsaturated groups per molecule. More preferably, the linear or branched hydrocarbon moiety will have at least 18 carbon atoms and at least two unsaturated groups per molecule.

The polyunsaturated fatty ester additive may include a polyunsaturated fatty acid. Preferably, the polyunsaturated fatty acid is a fatty acid compound having at least 18 carbon atoms and at least two unsaturated groups per molecule. The polyunsaturated fatty acid may be selected from an unsaturated fatty acid and a salt or ester thereof. The unsaturated fatty acid compound is not necessarily a pure substance, and the unsaturated fatty acid and the salt or ester thereof may contain a substituent such as a hydroxyl group or a formyl group. Some exemplary embodiments of the unsaturated fatty acid and compound thereof are oleic acid, linoleic acid, arachidonic acid, parinaric acid, dimer acid, ricinoleic acid, and fats and oils containing triglyceride thereof, esters thereof, or transition metal salts thereof. In certain embodiments, the transition metal salts of unsaturated fatty acids can be also used as a transition metal catalyst.

Another embodiment of the present invention includes a wall for a package having at least one layer. The layer is made of a composition including a substantially antimony-free and substantially phosphorus-free polyester base polymer; an oxidizable polyether-based additive; a transition metal catalyst, a colorant, and a polyunsaturated fatty ester additive having at least two unsaturated groups per molecule. The unsaturated groups may be carbon-carbon double bonds. Preferred compounds for the polyester base polymer, the oxidizable polyether-based additive, and the transition metal catalyst are as described above.

### BRIEF DESCRIPTION OF THE DRAWINGS

FIG. 1 is a graph depicting oxygen ingress data for a PET bottle according to Control Example 1, Example 1, and Comparative Example 1;

FIG. 2 is a graph depicting oxygen ingress data for PET bottles according to Control Example 2 and Example 2:

FIG. 3 is a graph depicting oxygen ingress data for PET bottles according to Control Example 3 and Example 3; and

FIG. 4 is a graph depicting oxygen ingress data for a PET bottle according to Control Example 4 and Example 4, and Example 5.

### DETAILED DESCRIPTION OF THE INVENTION

Embodiments of the present invention include compositions that are useful in the manufacture of packaging for oxygen-sensitive materials. In some embodiments, the composition includes a polyester base polymer, an oxidizable polyether-based additive, a transition metal salt as an oxidation catalyst, a colorant, and a polyunsaturated fatty ester additive having at least two unsaturated groups per molecule, wherein the unsaturated groups may be carbon-carbon double bonds. In preferred embodiments, the polyester base polymer is substantially free of antimony and substantially free of phosphorous, and wherein the composition exhibits excellent oxygen scavenging properties as well as excellent clarity (i.e., lack of haze) when blow molded, for example, from a preform into, for example, a monolayer container via an injection stretch blow molding process.

If the polyester base polymer contains unacceptably high levels of antimony or phosphorous, the composition requires an induction period prior to any significant oxygen scavenging. While not being bound by any specific theory, it is believed that initially the small amount of oxygen that permeates into the wall of a preform or bottle made from the composition reacts with the transition metal salt to form peroxide-free radicals believed to be needed for the initiation and propagation of free radical oxidation chain reaction on the polyether additive thus triggering the oxygen scavenging in the preform or bottle. Depending on the presence of any inhibitor impurities in the PET such as antimony or phosphorous, the catalytic activity of the transition metal catalyst as well as the free radical initiation and propagation may be deactivated to a varying extent, resulting in an induction period before the onset of oxygen scavenging. Accordingly, by maintaining a sufficiently low concentration of antimony and phosphorus, a bottle may be formed without any significant induction period. In addition to the polyester base polymer, each of the polyester base polymer, the oxidizable polyether-based additive, the transition metal salt, the colorant, and the polyunsaturated fatty ester additive having at least two unsaturated groups per molecule will now be described in greater detail.

### 1) Polyester Base Polymer

In preferred embodiments, the base polymer is a polyester. Examples of suitable polyester polymers include polyethylene terephthalate homopolymers and copolymers of polyethylene terephthalate modified with one or more polycarboxylic acid modifiers and hydroxyl compound modifiers (collectively, “PET”), polyethylene naphthalate homopolymers and copolymers of polyethylene naphthalate modified with one or more polycarboxylic acid modifiers and hydroxyl compound modifiers (“PEN”), and blends of PET and PEN. A suitable PET or PEN polymer may include the one or more polycarboxylic acid modifiers in a cumulative amount of less than about 15 mole %, or less than about 10 mole %, or less than about 8 mole %. A suitable PET or PEN polymer may include the one or more hydroxyl compound modifiers in an amount of less than about 60 mole %, or less than about 50 mole %, or less than about 40 mole %, or less than about 15 mole %, or less than about 10 mole %, or less than about 8 mole %. A modifier polycarboxylic acid compound or hydroxyl compound is a compound other than the compound contained in an amount of at least about 85 mole %. The preferred polyester polymer is polyalkylene terephthalate, and most preferred is PET. In some embodiments, the polyester polymer contains at least about 90 mole % ethylene terephthalate repeat units, and in other embodiments, at least about 92 mole %, and in yet other embodiments at least about 94 mole %, based on the moles of all repeat units in the polyester polymers.

In addition to a diacid component of terephthalic acid, derivatives of terephthalic acid, naphthalene-2,6-dicarboxylic acid, derivatives of naphthalene-2,6-dicarboxylic acid, or mixtures thereof, the polycarboxylic acid component(s) of the present polyester may include one or more additional modifier polycarboxylic acids. Such additional modifier polycarboxylic acids include aromatic dicarboxylic acids preferably having about 8 to about 14 carbon atoms, aliphatic dicarboxylic acids preferably having about 4 to about 12 carbon atoms, or cycloaliphatic dicarboxylic acids preferably having about 8 to about 12 carbon atoms.

Examples of modifier dicarboxylic acids useful as an acid component(s) are phthalic acid, isophthalic acid, naphthalene-2,6-dicarboxylic acid, cyclohexanedicarboxylic acid, cyclohexanediacetic acid, diphenyl-4,4′-dicarboxylic acid, succinic acid, glutaric acid, adipic acid, azelaic acid, sebacic acid, and the like, with isophthalic acid, naphthalene-2,6-dicarboxylic acid, and cyclohexanedicarboxylic acid being most preferable. It should be understood that use of the corresponding acid anhydrides, esters, and acid chlorides of these acids is included in the term “polycarboxylic acid.” It is also possible for trifunctional and higher order polycarboxylic acids to modify the polyester.

The hydroxyl component is made from compounds containing 2 or more hydroxyl groups capable of reacting with a carboxylic acid group. In some preferred embodiments, preferred hydroxyl compounds contain 2 or 3 hydroxyl groups. Certain preferred embodiments have 2 hydroxyl groups. These hydroxyl compounds include C₂-C₄ alkane diols, such as ethylene glycol, propane diol, and butane diol, among which ethylene glycol is most preferred for container applications. In addition to these diols, other modifier hydroxyl compound component(s) may include diols such as cycloaliphatic diols preferably having 6 to 20 carbon atoms and/or aliphatic diols preferably having about 3 to about 20 carbon atoms. Examples of such diols include diethylene glycol; triethylene glycol; 1,4-cyclohexanedimethanol; propane-1,3-diol and butane-1,4-diol (which are considered modifier diols if ethylene glycol residues are present in the polymer in an amount of at least 85 mole % based on the moles of all hydroxyl compound residues); pentane-1,5-diol; hexane-1,6-diol; 3-methylpentanediol-(2,4); neopentyl glycol; 2-methylpentanediol-(1,4); 2,2,4-trimethylpentane-diol-(1,3); 2,5-ethylhexanediol-(1,3); 2,2-diethyl propane-diol-(1,3); hexanediol-(1,3); 1,4-di-(hydroxyethoxy)-benzene; 2,2-bis-(4-hydroxycyclohexyl)-propane; 2,4-dihydroxy-1,1,3,3-tetramethyl-cyclobutane; 2,2-bis-(3-hydroxyethoxyphenyl)-propane; and 2,2-bis-(4-hydroxypropoxyphenyl)-propane. Typically, polyesters such as polyethylene terephthalate are made by reacting a glycol with a dicarboxylic acid as the free acid or its dimethyl ester to produce an ester monomer and/or oligomers, which are then polycondensed to produce the polyester.

In some preferred embodiments, modifiers include isophthalic acid, naphthalenic dicarboxylic acid, trimellitic anhydride, pyromellitic dianhydride, 1,4-cyclohexane dimethanol, and diethylene glycol. The amount of the polyester polymer in the formulated polyester polymer composition ranges from greater than about 50.0 wt. %, or greater than about 80.0 wt. %, or greater than about 90.0 wt. %, or greater than about 95.0 wt. %, or greater than about 96.0 wt. %, or greater than about 97.0 wt. %, and up to about 99.90 wt. %, based on the combined weight of all polyester polymers and all polyamide polymers. The formulated polyester polymer compositions may also include blends of formulated polyester polymer compositions with other thermoplastic polymers such as polycarbonate. In some preferred compositions, the polyester comprises a majority of the composition of the inventions, and in some embodiments the polyester is present in an amount of at least about 80 wt. %, or at least about 90 wt. %, based on the weight of the composition (including the oxidizable polyether-based additive and a transition metal salt, but excluding fillers, inorganic compounds or particles, fibers, impact modifiers, or other polymers which serve as impact modifiers or which form a discontinuous phase such as may be found in cold storage food trays).

The polyester base polymer is substantially free of antimony. In one embodiment, the term “substantially free of antimony” refers to polyester base polymers including less than about 100 ppm of antimony, preferably less than about 50 ppm, more preferably less than about 10 ppm, and most preferably from about 0 ppm to about 2 ppm. In another embodiment, the term “substantially free of antimony” refers to polyester base polymers comprising 46 ppm or less of antimony, preferably 40 ppm or less of antimony, more preferably 31.4 ppm or less of antimony, and most preferably 15.7 ppm or less of antimony. It is also preferable that the base polymer is substantially free of phosphorus. As used herein, the term “substantially free of phosphorus” refers to polyester base polymers including less than about 20 ppm of phosphorus, preferably less than about 10 ppm, more preferably less than about 5 ppm, and most preferably the polyester base polymer includes about 0 ppm to about 1 ppm. PET polymers formed using typical antimony metal-based catalysts typically contain about 190 ppm to about 300 ppm antimony and about 20 ppm to about 100 ppm of phosphorus.

In an exemplary embodiment, the antimony-free polyester base polymer is selected from PET resins formed using titanium, germanium, or aluminum metal-based catalysts. In some embodiments, the polyester base polymer may include a blend of a low-antimony or substantially antimony-free polyester base polymer and a polyester base polymer having a greater concentration of antimony, so long as the blend has an antimony concentration below the limits described above. Examples of preferred antimony-free PET resins are selected from titanium catalyst-based PET resins such as Laser+® HS Ti 818, W Ti 844 and the aluminum catalyst-based PET resins such as Laser+® B92A (formerly Parastar 7000) available from DAK America. The polyester base polymer may preferably have an intrisic viscosity (IV) ranging from about 0.5 dl/g to about 1.0 dl/g, more preferably from about 0.65 dl/g to about 0.9 dl/g and most preferably from about 0.72 dl/g to about 0.84 dl/g.

Other base polymers may be used with the instant invention provided that the other base polymer also has a sufficiently low level of antimony. One example is polypropylene. In certain embodiments, the polyester polymers of the invention are thermoplastic and, thus, the form of the compositions are not limited and can include a composition in the melt phase polymerization, as an amorphous pellet, as a solid stated polymer, as a semi-crystalline particle, as a composition of matter in a melt processing zone, as a bottle preform, or in the form of a stretch blow molded bottle or other articles.

### 2) Oxidizable Polyether-Based Additive

In preferred embodiments, the oxidizable polyether-based additive includes the general structure of:

X—[R—O]ₙ—R′—Y,

wherein R is a substituted or unsubstituted alkylene chain having from 2 to 10 carbon atoms (such as ethylene, propylene, butylene, 1,4-tetramethylene, etc.):

n ranges from 4 to 100;

X and Y are selected from H, OH, —OCOR₁ groups, —OCOAr₁, —OR₁, and —OAr₁ groups, where R₁ is an alkyl group (such as methyl, ethyl, propyl and so on up to C18) and Ar is an aryl group (such as an unsubstituted or substituted phenyl, naphthyl, etc.); and

R′ may be the same as R or selected from the group consisting of
—[COR₂COOR₃O]ₚ— and —[COAr₂COOR₃O]ₚ—, wherein Ar₂ is a phenylene or naphthylene group, R₂ and R₃ are C₂ to C₁₈ alkylene groups, and p ranges from 10 to 100.

As used herein, the term “alkyl” refers to a substituted or unsubstituted aliphatic hydrocarbon chain. Alkyl groups have straight and branched chains. In some embodiments, alkyls have from 1 to 12 carbon atoms or 1 to 6 carbon atoms, unless explicitly specified otherwise. Alkyl groups include, but are not limited to methyl, ethyl, propyl, isopropyl, butyl, 1-butyl and t-butyl. Specifically included within the definition of “alkyl” are those aliphatic hydrocarbon chains that are optionally substituted.

As used herein, the term “alkylene” or “alkylenyl” means a divalent alkyl linking group. Example of alkylenes (or alkylenyls) include, but are not limited to, methylene or methylenyl (—CH₂—), ethylene or ethylenyl (—CH₂—CH₂—), and propylene or propylenyl (—CH₂—CH₂—CH₂—).

As used herein, the term “aryl” is defined herein as an aromatic carbocyclic moiety of up to 20 carbon atoms. In some embodiments, aryl groups have 6-20 carbon atoms or 6-14 carbon atoms. Aryls may be a single ring (monocyclic) or multiple rings (bicyclic, up to three rings) fused together or linked covalently. Any suitable ring position of the aryl moiety may be covalently linked to the defined chemical structure. Aryl groups include, but are not limited to, phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, and acenaphthylenyl. In some embodiments, phenyl is a preferred aryl. Aryl groups may also be optionally substituted with one or more substituents.

Optional substituents for alkyl, alkenyl, or aryl groups are well known to those skilled in the art. These substituents include alkyl, alkoxy, aryloxy, hydroxy, acetyl, cyano, nitro, glyceryl, and carbohydrate, or two substituents taken together may be linked as an alkylene group to form a ring.

The preferred polyether based additives are selected from:
(1) polyether diols (also known as polyols) such as polyethylene glycol, polypropylene glycol, polytetramethylene ether glycol (PTMEG), of which PTMEG is preferred;
(2) ester end-capped derivatives of polyether diols (i.e., ∝,ω-polyether diesters), of which PTMEG diesters are preferred, and PTMEG dibenzoate or dioctaoate are most preferred;
(3) polyether-polyester block copolymers such as PTMEG-b-PET, PTMEG-b-PBT copolymers, of which PTMEG-b-PET copolymer in which the PTMG content is at least 40 wt. % is preferred; and
(4) ether end-capped derivatives of polyetherdiols (e.g., μ,ω-polyether diethers) of which PTMEG diethers are preferred, and PTMEG-μ,ω-dimethyl ether or PTMEG-μ,ω-diethyl ether are the most preferred.

In an embodiment where a PET container such as a monolayer bottle is made from the composition, the polyether-based additive may include up to about 5 wt. % of the bottle, preferably at least 0.5 wt. %. For example, an exemplary bottle may include up to about 1 wt. %, about 2 wt. %, about 3 wt. %, about 4 wt. % or about 5 wt. % of the polyether-based additive (depending on the thickness of the layer). Conversely, the composition may include between about 0.5 wt. % and about 2 wt. % of the polyether-based additive. In another embodiment, the composition may include at least 0.5 wt. %, and typically about 1 wt. % to about 5 wt. % (depending on the thickness of the layer).

### 3) Transition Metal Salt

The instant compositions include as an oxidation catalyst a transition metal salt including a metal in a positive oxidation state. It should be noted that it is contemplated that one or more such metals may be used. The transition metal functions to catalyze or promote the oxidation of the organic oxidizable component (i.e., the reaction of the oxidizable polyether-based additive with molecular oxygen).

The transition metal can be selected from the first, second, or third transition series of the Periodic Table. The metal can be Rh, Ru, or one of the elements in the series of Sc to Zn (i.e., Sc, V, Cr, Mn, Fe, Co, Ni, Cu, and Zn). In some embodiments, cobalt is added in +2 or +3 oxidation state. In some embodiments, it is preferred to use cobalt in the +2 oxidation state. In certain embodiments, copper in the +2 oxidation state is utilized. In some embodiments, rhodium in the +2 oxidation state is used. In certain embodiments, zinc may also be added to the composition. Preferred zinc compounds include those in a positive oxidation state.

Suitable counter-ions to the transition metal cations include carboxylates, such as neodecanoates, octanoates, acetates, lactates, naphthalates, malates, stearates, acetylacetonates, linoleates, oleates, palmitates, 2-ethylhexanoates, or ethylene glycolates; or as their oxides, borates, carbonates, chlorides, dioxides, hydroxides, nitrates, phosphates, sulfates, or silicates among others.

In a preferred embodiment, the transition metal catalyst is selected from any cobalt carboxylate salt, preferably cobalt salts of C₂ to C₁₈ carboxylic acids. Most preferably, the transition metal catalyst is a pastille-form cobalt neodecanoate composed of a mixture of cobalt propionate and cobalt neodecanoate.

In some embodiments, the composition has a transitional metal concentration of about 20 ppm to about 400 ppm, preferably about 30 ppm to about 200 ppm, and most preferably about 50 ppm to about 100 ppm. The exact amount of transition metal used in an application can be determined by trials that are well within the skill level of one skilled in the art.

The transition metal or metals may be added neat or in a carrier (such as a liquid or wax) to an extruder or other device for making the article, or the metal may be present in a concentrate or carrier with the oxidizable organic component, in a concentrate or carrier with a base polymer, or in a concentrate or carrier with a blend of the base polymer and oxidizable polyether-based additive. Alternatively, at least a portion of the transition metal may be added as a polymerization catalyst to the melt phase reaction for making the base polymer (a polyester polymer in some embodiments) and be present as residual metals when the polymer is fed to the melting zone (e.g. the extrusion or injection molding zone) for making the article such as a preform or sheet. It is desirable that the addition of the transition metal does not substantially increase the IV of the melt in the melt processing zone. Thus, transition metal or metals may be added in two or more stages, such as once during the melt phase for the production of the polyester polymer and again once more to the melting zone for making the article.

### 4) Colorant

The instant compositions include at least one colorant. Suitable colorants include any of the organic dyes, organic pigments, inorganic dyes and inorganic pigments that are typically used as colorants in polymer applications. Examples of such colorants include the following colorants of respective colors to be shown below. In the following, the designation “C. I.” means color index.

A black colorant includes, for example, carbon black, copper oxide, manganese dioxide, aniline black, activated carbon, non-magnetic ferrite, magnetic ferrite, and magnetite.

A yellow pigment includes, for example, C.I. pigment yellow 13, C. I. pigment yellow 14, C. I. pigment yellow 17, C. I. pigment yellow 74, C. I. pigment yellow 93, C. I. pigment yellow 155, C. I. pigment yellow 180, and C. I. pigment yellow 185.

An orange colorant includes, for example, red chrome yellow, molybdenum orange, permanent orange GTR, pyrazolone orange, vulcan orange, indathrene brilliant orange RK, benzidine orange G, indathrene brilliant orange GK, C.I. pigment orange 31, C. I. pigment orange 43.

A red colorant includes, for example, C.I. pigment red 52, C.I. pigment red 53, C. I. pigment red 19, C.I. pigment red 48:1, C.I. pigment red 48:2, C. I. pigment red 48:3, C. I. pigment red 57:1, C. I. pigment red 122, C. I. pigment red 150, and C. I. pigment red 184.

The colorants can be used each alone or two or more of them of different colors can be used together. A plurality of colorants of an identical color system can also be used together. The ratio of the colorant used to the polyester based polymer is not particularly restricted and can be properly selected within a wide range in accordance with various conditions such as the type of polyester based polymer the colorant, the characteristics required for the desired color to be achieved. As an example, the ratio of the colorant used to the polyester based polymer binder resin can be preferably from 0.01 part by weight or 1 parts by weight or less, and more preferably, 0.2 parts by weight or more and 0.5 parts by weight or less based on 100 parts by weight of the polyester based polymer.

The addition of at least one colorant results in the observance of an induction period where, in the absence of the colorant, an induction period is not observed. While not being bound by any specific theory, it is believed that the conjugated aromatic structure of the colorant (typically anthraquinone, aromatic azo or quinacridone type structures) causes free radical trapping reactions and hence oxygen scavenging inhibition resulting in an increased induction period. To address this increased induction period brought about by the incorporation of a colorant a second oxygen scavenger is incorporated.

### 5) Polyunsaturated Fatty Ester Additives

The compostions disclosed herein comprise at least one polyunsaturated fatty ester oil additive including a polyunsaturated fatty ester with the formula R—[OCOCₓH_{y}]ₙ wherein, R is an alkyl, a alkylene, a trivalent alkane group, or a glyceryl moiety; n ranges from 1 to 3; x ranges from 16 to 20; and y ranges from 27 to 35. In a preferred embodiment, R is a glyceryl moiety, n=3, x=18, and y=33 to 35. Examples of polyunsaturated fatty ester additives are linoleic acid, linoelaidic acid, α-linolenic acid, and arachidonic acid.

Preferably, the polyunsaturated fatty ester oil additive will have an unsaturated fatty ester content of at least 80%. More preferably, the polyunsaturated fatty ester oil additive will have an unsaturated fatty ester content of greater than 90%. Preferably, the polyunsaturated fatty ester oil additive will have a polyunsaturated fatty ester content of at least 50%. More preferably, the polyunsaturated fatty ester oil additive will have a polyunsaturated fatty ester content of greater than 75%. Examples of such polyunsaturated fatty ester oil additive include, for example, corn oil, cottonseed oil, flaxseed/linseed oil, grapeseed oil, hemp oil, pumpkin seed oil, safflower oil, soybean oil, sunflower oil, or walnut oil.

The at least one polyunsaturated fatty ester oil additive including a polyunsaturated fatty ester as defined above is not necessarily a pure substance, and may contain a substituent such as a hydroxyl group or a formyl group. In this regard, fatty esters are natural products, which has the consequence, that they consist of a mixture of various chain lengths, with the emphasis on the indicated value, (i.e. a C₁₈ chain will accordingly also contain, apart from the majority of C₁₈, also amounts of C₁₆ and C₂₀, or even some C₁₄ or C₂₂). Thus, the chain length indicated for the polyunsaturated fatty ester additives is to be understood in the accepted sense in the art, namely that of a mixture of chain lengths distributed around the indicated value, with the chain length indicated being present as the largest fraction.

The amounts of the components used in the oxygen scavenging formulations of the present invention can affect the use and effectiveness of this composition. Thus, the amounts of polyester base polymer, oxidizable polyether-based additive, transition metal salt, colorant, and polyunsaturated fatty ester additives can vary depending on the desired article and its end use. For example, a primary function of the organic oxidizable components detailed above is to react irreversibly with oxygen during the scavenging process, while a primary function of the transition metal catalyst is to facilitate this process. Thus, to a large extent, the amount of the organic oxidizable component present affects the oxygen scavenging capacity of the composition, i.e., the amount of oxygen that the composition can consume, while the amount of transition metal catalyst affects the rate at which oxygen is consumed as well as the induction period.

It has been found that the use of a minor amount of polyunsaturated fatty ester additives overcomes the inhibitive effect on oxygen scavenging of colorants in monolayer substantially antimony-free and substantially-phosphorous-free PET containers made with an oxidizable polyether-based additive.

In an embodiment where a PET container such as a monolayer bottle is made from the composition, the polyunsaturated fatty ester oil additive including the polyunsaturated fatty ester additives may be up to about 1 wt. % of the bottle, preferably at least 0.1 wt. %. For example, an exemplary bottle may include up to about 0.2 wt. %, about 0.3 wt. %, about 0.4 wt. %, about 0.5 wt. % about 0.6 wt. %, about 0.7 wt. %, about 0.8 wt. %, about 0.9 wt. %, or about 1.0 wt. % of the polyunsaturated fatty ester oil additive, including the polyunsaturated fatty ester additives. (depending on the thickness of the layer). Preferably, the composition can include between about 0.2 wt. % and about 0.8 wt. % of the polyunsaturated fatty ester oil additive, including the polyunsaturated fatty ester additives. More preferably, the composition can include between about 0.3 wt. % and about 0.5 wt. % of the polyunsaturated fatty ester oil additive, including the polyunsaturated fatty ester additives (depending on the thickness of the layer).

In an embodiment where a PET container such as a monolayer bottle is made from the composition, preferably the total amount of polyether-based additives and polyunsaturated fatty ester oil additive does not exceed about 5.0 wt. %. More preferably the total amount of polyether-based additives and polyunsaturated fatty ester oil additive is in the range of 1.0 wt. % to 5.0 wt. %. In these embodiments, the polyether-based additives are the main oxygen scavenger component. While not being bound by any theory it is believed that the higher reactivity of the polyunsaturated fatty ester additives in free radical initiation step overcomes the inhibiting effect of the colorants in the initial stages of oxygen scavenging, whereas the high scavenging capacity and steady-state reactivity of the polyether-based additives, ensures the long-term oxygen scavenging process and long product shelf life.

The oxygen scavenger composition of the present invention can be incorporated in packaging articles having various forms. Suitable articles include, but are not limited to, flexible sheet films, flexible bags, pouches, semi-rigid and rigid containers such as bottles (e.g., PET bottles) or metal cans, or combinations thereof.

Typical rigid or semi-rigid articles include plastic, paper or cardboard containers, such as those utilized for juices, soft drinks, as well as thermoformed trays or cups normally having a thickness in the range of from about 100 micrometers to about 1000 micrometers. The walls of such articles comprise single layers of materials. The articles can also take the form of a bottle or can, or a crown, cap, crown or cap liner, plastisol or gasket. The oxygen scavenger composition of the present invention can be used as an integral layer or portion of, or as an external or internal coating or liner of, the formed semi-rigid or rigid packaging article. As a liner, the oxygen scavenger composition can be extruded as a film along with the rigid article itself, in, e.g., a coextrusion, extrusion coating, or extrusion lamination process, so as to form the liner in situ during article production; or alternatively can be adhered by heat and/or pressure, by adhesive, or by any other suitable method to an outer surface of the article after the article has been produced.

In one preferred embodiment of the present invention, the composition of the present invention, i.e., a substantially antimony-free and substantially phosphorus-free polyester base polymer, a transition metal in a positive oxidation state, at least one oxidizable polyether-based additive as described above, a colorant and at least one polyunsaturated fatty ester additive can be employed to form a monolayer bottle.

Besides articles applicable for packaging food and beverage, articles for packaging other oxygen-sensitive products can also benefit from the present invention. Such products would include pharmaceuticals, oxygen sensitive medical products, corrodible metals or products, electronic devices and the like.

The composition may also include other components such as fillers, crystallization aids, impact modifiers, surface lubricants, denesting agents, stabilizers, ultraviolet light absorbing agents, metal deactivators, nucleating agents such as polyethylene and polypropylene, and phosphite stabilizers. Other additional components are well known to those skilled in the art and can be added to the existing composition so long as they do not negatively impact the performance of the compositions. Typically, the total quantity of such components will be less than about 10% by weight relative to the total composition. In some embodiments, the amount of these optional components is less than about 5%, by weight relative to the total composition.

A common additive used in the manufacture of polyester polymer compositions used to make stretch blow molded bottles is a reheat additive because the preforms made from the composition must be reheated prior to entering the mold for stretch blowing into a bottle. Any of the conventional reheat additives can be used, such additives include various forms of black particles, e.g. carbon black, activated carbon, black iron oxide, glassy carbon, and silicon carbide; and other reheat additives such as silicas, red iron oxide, and so forth.

In many applications, not only are the packaging contents sensitive to the ingress of oxygen, but the contents may also be affected by UV light. Fruit juices and pharmaceuticals are two examples of such contents. Accordingly, in some embodiments, it is desirable to incorporate into the polyester composition any one of the known UV-absorbing compounds in amounts effective to protect the packaged contents.

The instant compositions can be made by mixing a substantially antimony-free and substantially phosphorous-free polyester base polymer (PET, for example) with the oxidizable polyether-based additive and the transition metal catalyst. Such compositions can be made by any method known to those skilled in the art. In certain embodiments, some or part of the transition metal of the transition metal catalyst may exist in the base polymer prior to mixing. This residual metal, for example, can exist from the manufacturing process of the base polymer. In some embodiments, the substantially antimony-free and substantially phosphorous-free polyester base polymer, the oxidizable polyether-based additive and the transition metal catalyst are mixed by tumbling in a hopper. Other optional ingredients can be added during this mixing process or added to the mixture after the aforementioned mixing or to an individual component prior to the aforementioned mixing step.

The instant composition can also be made by adding each ingredient separately and mixing the ingredients prior melt processing the composition to form an article. In some embodiments, the mixing can be just prior to the melt process zone. In other embodiments, one or more ingredients can be premixed in a separate step prior to bringing all of the ingredients together.

In some embodiments, the invention concerns use of the compositions described herein as a component of a wall that is used in a package for oxygen sensitive materials. The necessary scavenging capacity of a package will generally have to be greater for walls that have a greater permeance in the absence of scavenging additives. Accordingly, a good effect is harder to achieve when inherently higher permeance materials are used.

The wall may be a rigid one, a flexible sheet, or a clinging film. It may be homogenous or a laminate or coated with other polymers. If it is laminated or coated, then the scavenging property may reside in a layer of the wall the permeance of which is relatively high in the absence of scavenging and which alone would not perform very satisfactorily but which performs satisfactorily in combination with one or more other layers which have a relatively low permeance but negligible or insufficient oxygen-scavenging properties. A single such layer could be used on the outside of the package since this is the side from which oxygen primarily comes when the package is filled and sealed. However, such a layer to either side of the scavenging layer would reduce consumption of scavenging capacity prior to filling and sealing.

When the instant compositions are used in a wall or as a layer of a wall, the permeability of the composition for oxygen is advantageously not more than about 3.0, or not more than about 1.7, or not more than about 0.7, or not more than about 0.2, or not more than about 0.03 cm³ mm/(m² atm day). The permeability of the composition provided by the present invention is advantageously not more than about three-quarters of that in the absence of oxygen-scavenging properties. In some embodiments, the permeability is not more than about one half, one-tenth in certain embodiments, one twenty-fifth in other embodiments, and not more than one-hundredth in yet other embodiments of that in the absence of oxygen-scavenging properties. The permeability in the absence of oxygen-scavenging properties is advantageously not more than about 17, or not more than about 10, or not more than about 6 cm³ mm/(m² atm day). A particularly good effect can be achieved for such permeabilities in the range from about 0.5, or about 1.0, to 10, or about 6.0, cm³ mm/(m² atm day). Measuring oxygen permeation can be performed by one of ordinary skill in the art employing oxygen permeation (OTR) instrumentation such as, for example, OX-TRAN® instruments available from MOCON. Inc. (Minneapolis, Minn.).

The above-described permeabilities are achieved without an induction period, which, in practical terms means that such permeabilities are achievable immediately after the container is formed.

In another aspect, the instant composition can be used as a master batch for blending with a polymer or a polymer containing component. In such compositions, the concentration of the oxidizable polyether-based additive and the transition metal catalyst will be higher to allow for the final blended product to have suitable amounts of these components. The master batch may also contain an amount of the polymer to which the master batch is to be blended with. In other embodiments, the master batch may contain a polymer that is compatible with the polymer to which the master batch is to be blended.

The time period for which the permeability is maintained can be extended by storing the articles in sealed containers or under an inert atmosphere such as nitrogen prior to use with oxygen sensitive materials.

In another aspect, the invention provides a package, whether rigid, semi-rigid, collapsible, lidded, or flexible or a combination of these, comprising a wall as formed from the compositions described herein. Such packages can be formed by methods well known to those skilled in the art.

Among the techniques that may be used to make articles are molding generally, injection molding, stretch blow molding, extrusion, thermoforming, and extrusion blow molding. Orientation, e.g., by stretch blow molding, of the polymer is especially attractive with phthalate polyesters because of the known mechanical advantages that result.

The melt processing zone for making the article can be operated under customary conditions effective for making the intended articles, such as preforms, bottles, trays, and other articles mentioned below. In one embodiment, such conditions are effective to process the melt without substantially increasing the IV of the melt and which are ineffective to promote transesterification reactions. In some preferred embodiments, suitable operating conditions effective to establish a physical blend of the substantially antimony-free polyester polymer, oxidizable polyether-based additive, and transition metal catalyst are temperatures in the melt processing zone within a range of about 250° C. to about 300° C. at a total cycle time of less than about 6 minutes, and typically without the application of vacuum and under a positive pressure ranging from about 0 psig to about 900 psig. In some embodiments, the residence time of the melt on the screw can range from about 1 to about 4 minutes.

Specific articles include preforms, containers and films for packaging of food, beverages, cosmetics, pharmaceuticals, and personal care products where a high oxygen barrier is needed. Examples of beverage containers are bottles for holding water and carbonated soft drinks, and the invention is particularly useful in bottle applications containing juices, sport drinks, beer or any other beverage where oxygen detrimentally affects the flavor, fragrance, performance (prevent vitamin degradation), or color of the drink. The compositions of the instant invention are also particularly useful as a sheet for thermoforming into rigid packages and films for flexible structures. Rigid packages include food trays and lids. Examples of food tray applications include dual ovenable food trays, or cold storage food trays, both in the base container and in the lidding (whether a thermoformed lid or a film), where the freshness of the food contents can decay with the ingress of oxygen. The compositions of the instant invention also find use in the manufacture of cosmetic containers and containers for pharmaceuticals or medical devices.

In this specification and in the claims that follow, reference will be made to a number of terms, which shall be defined to have the following meanings.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “a”, “an”, “the” and the like refer to both the singular and plural unless the context clearly indicates otherwise. “A bottle”, for example, refers to a single bottle or more than one bottle.

Also as used herein, the description of one or more method steps does not preclude the presence of additional method steps before or after the combined recited steps. Additional steps may also be intervening steps to those described. In addition, it is understood that the lettering of process steps or ingredients is a convenient means for identifying discrete activities or ingredients and the recited lettering can be arranged in any sequence.

Where a range of numbers is presented in the application, it is understood that the range includes all integers and fractions thereof between the stated range limits. A range of numbers expressly includes numbers less than the stated endpoints and those in-between the stated range. A range of from 1-3, for example, includes the integers one, two, and three as well as any fractions that reside between these integers.

As used herein, “master batch” refers to a mixture of base polymer, oxidizable organic component, and transition metal that will be diluted, typically with at least additional base polymer, prior to forming an article. As such, the concentrations of oxidizable organic component and transition metal are higher than in the formed article.

The following examples are included to demonstrate preferred embodiments of the invention regarding the usefulness of low-antimony low phosphorous PET base resin blended with an oxidizable polyether-based additive and a transition metal salt catalyst to make oxygen scavenging, clear PET containers which exhibit no induction period. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

In the following examples, various compositions of PET resin blended with mixtures of OS additives, and/or transition metal catalysts were fabricated into monolayer bottles via a 2-step process. In the 1^{st} step, each composition was directly fed and melt-blended in an injection molding machine and then molded into the preforms. In a 2^{nd} step, the preforms were reheated and stretch blow molded into the final shaped containers such as bottles.

The monolayer preforms were made on a single cavity, 2003 Battenfeld A800/200H/125HC injection molding machine. The blended composition was fed into the throat of the injection molding extruder heated to 260-270° C. The molten blend was then injection molded into a single cavity preform mold, such as a 30 g, 33 mm finish 20 oz. ketchup bottle preform, to form the monolayer bottle preform. The cycle time for molding was about 30 sec. The preforms were then reheat-stretch-blow molded into monolayer bottles. The bottles were generally stretch blown on a Side1 SBO-1 machine set to run at a rate of ca. 800 bottles per hour. In this process, the preforms were typically heated to a surface temperature of 99° C. prior to the blowing operation. The blow mold temperature was about 12° C. The blow pressures were about 33 bar. Clear monolayer PET blend bottles were thus obtained.

The oxygen scavenging performance of all the PET bottles from examples were evaluated using an Oxysense 4000B instrument with OxyDot oxygen sensors (available from OxySense Inc. Dallas, Tex. 752543, USA), for the measurement of oxygen ingress/oxygen content with time. Typically the OxyDots were attached to the inside middle portion of each test bottle. Each bottle is then loaded on an orbisphere bench top filler and after an initial flushing with nitrogen, it is filled with deoxygenated water (O₂ content <100 ppb) and capped. After several bottles of each composition have been filled and sealed, they are stored under ambient conditions for a required shelf-life test period while the oxygen content or ingress in the bottles is measured. To make the measurements, the fiber optic pen of the instrument was aligned with the OxyDot (from the outside of the bottle), making sure that the tip of the pen was making contact with the bottle. Then the capture button was pressed to obtain the oxygen concentration in the bottle. The oxygen concentration was measured periodically with time.

### Comparative Example 1

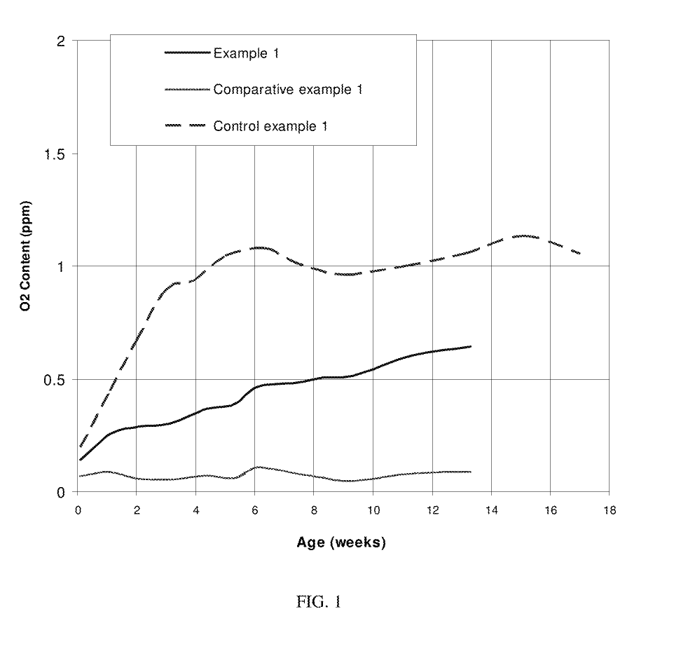

A dry blend of an antimony-free PET resin (Laser+HS Ti818, DAK America), herein after referred to as PET (Ti818), with 1 wt % of poly(tetramethylene ether)-PET block copolymer (Oxyclear® 3500, Auriga Polymer Inc.), hereinafter referred to as “OS-A” additive and 1 wt. % of a cobalt masterbatch in PET (Oxyclear® 2702, Auriga Polymers Inc, 1500 ppm Co) was made. This blend contains a total of 45 ppm cobalt (30 ppm of built-in Co from Ti818+15 ppm Co from the added 1% Oxyclear 2702) which serves the function of a catalyst for the oxygen scavenging. This dry blend was directly injection molded into preforms which were subsequently blown into monolayer 20 oz. ketchup bottles (512 ml volume, 0.04 cm sidewall thickness), using the 2 step process described before. The bottles were tested for oxygen scavenging performance using the Oxysense method described earlier. The oxygen ingress data for this example is shown in FIG. 1. These clear bottles without any colorant added, exhibited excellent oxygen scavenging performance.

### Control Example 1

A dry blend of PET (Ti818) with 1 wt % of “OS-A” additive, 1 wt. % of the cobalt masterbatch in PET (Oxyclear® 2702) hereinafter referred to as “Co-MB” and 0.7 w % Penn Red 66R8645 (an organic red colorant masterbatch from Penn Color, Doylestown, Pa.) was made. This dry blend was directly injection molded into preforms which were subsequently blown into monolayer 20 oz. ketchup bottles (512 ml volume, 0.04 cm sidewall thickness), using the 2 step process described before. These red colored monolayer bottles were tested for oxygen scavenging performance using the Oxysense method described earlier. The oxygen ingress data for this example is shown in FIG. 1. These red colored bottles contained the same amount of oxygen scavenger (1% OS-A) and catalyst (l %2702) as in above Comparative Example 1, but because of the added red colorant they showed an undesireable induction period of about 6 weeks before any oxygen scavenging started, resulting in >1 ppm of oxygen ingress in the 1^{st} 6 weeks in contrast to the clear bottles of Comparative Example 1.

### Example 1

A dry blend was made by thoroughly mixing PET (Ti818) resin pellets with 1 wt % of “OS-A” additive, 1 wt. % of Co-MB, 0.5 wt % Penn Red 66R8645 and 0.3 wt % of Flax seed oil (from a local store) as the OS-B additive. This dry blend was directly injection molded into preforms which were subsequently blown into monolayer 20 oz. ketchup bottles (512 ml volume, 0.04 cm sidewall thickness), using the 2 step process described before. These red colored monolayer bottles were tested for oxygen scavenging performance using the Oxysense method described earlier. The oxygen ingress data for this example is shown in FIG. 1. These red colored bottles containing just 0.3 wt % flax seed oil rich in the polyunsaturated fatty ester content, showed improved oxygen scavenging performance with reduced induction period.

### Control Example 2

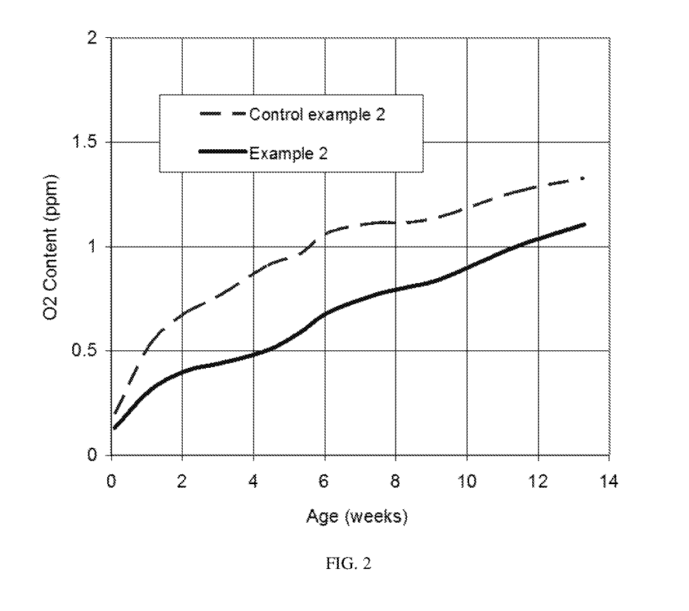

A dry blend of PET (Ti818) resin with 1 wt % of “OS-A” additive, 1 wt. % of Co-MB and 0.5 wt % Penn Chromatic Red (another organic red colorant masterbatch from Penn Color, Doylestown, Pa.) was made. This dry blend was directly injection molded into preforms which were subsequently blown into monolayer 20 oz. ketchup bottles using the 2 step process described before. These red colored monolayer bottles were tested for oxygen scavenging performance. The oxygen ingress data for this example is shown in FIG. 2. These red colored bottles also showed an induction period of ca. 6 weeks before the oxygen scavenging began, resulting in >1 ppm of oxygen ingress in the first 6 weeks.

### Example 2

A dry blend of PET (Ti818) resin with 1 wt % of “OS-A” additive, 1 wt. % of Co-MB, 0.5 wt % Penn Chromatic Red and 0.3 wt % flax seed oil was made. This dry blend was directly injection molded into preforms which were subsequently blown into monolayer 20 oz. ketchup bottles using the 2 step process described before. These red colored monolayer bottles were tested for oxygen scavenging performance. The oxygen ingress data for this example is shown in FIG. 2. These red colored bottles showed reduced induction period and improved oxygen scavenging performance relative to Control Example 2.

### Control Example 3

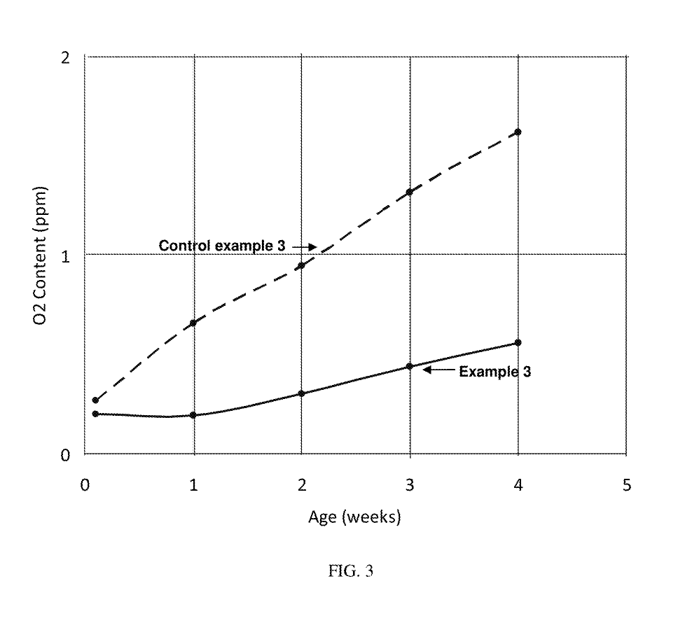

A dry blend of Oxyclear® 2512 resin (a specialty PET resin made with ca. 60 ppm built-in cobalt, from Auriga polymers, Indorama Ventures, Spartanburg, S.C.) hereinafter referred to as “2512 PET resin”, with 1 wt % of “OS-A” additive and 0.5 wt % PolyOne CC102665225P2RedV6 (an experimental organic red colorant hereinafter referred to as “PolyOne red” from PolyOne, Avon Lake, Ohio), was made. This dry blend was directly injection molded into preforms which were subsequently blown into monolayer 20 oz. ketchup bottles using the 2 step process described before. These red colored monolayer bottles were tested for oxygen scavenging performance. The oxygen ingress data for this example is shown in FIG. 3. These red colored bottles also showed an induction period for oxygen scavenging resulting in significant oxygen ingress (>1.6 ppm of oxygen in the first 4 weeks).

### Example 3

A dry blend of “2512 PET resin” with 1 wt % of “OS-A” additive and 0.5 wt % PolyOne red was made. This dry blend was directly injection molded into preforms which were subsequently blown into monolayer 20 oz. ketchup bottles using the 2 step process described before. These red colored monolayer bottles were tested for oxygen scavenging performance. The oxygen ingress data for this example is shown in FIG. 3. These red colored monolayer PET bottles showed improved oxygen scavenging performance relative to Control Example 3.

### Control Example 4

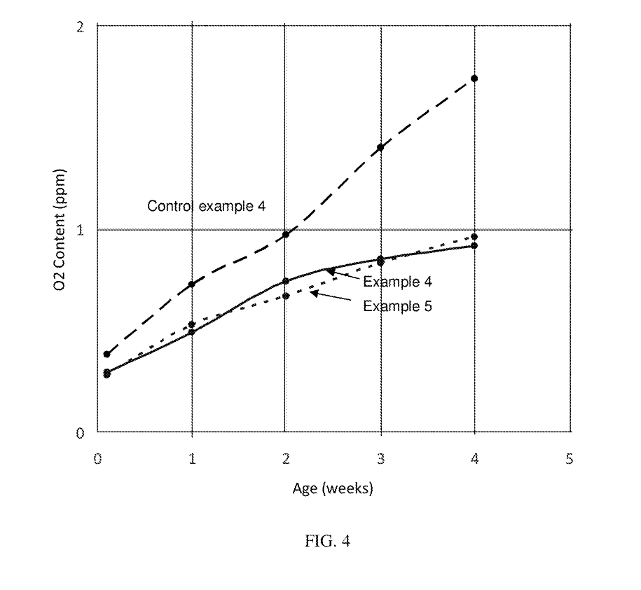

A dry blend of “2512 PET resin” with 1 wt % of “OS-A” additive and 0.6 wt % Penn Color Red 66R8980 (another red colorant masterbatch from Penn Color) was made. This dry blend was directly injection molded into preforms which were subsequently blown into monolayer 20 oz. ketchup bottles using the 2 step process described before. These red colored monolayer bottles were tested for oxygen scavenging performance. The oxygen ingress data for this example is shown in FIG. 4. It may be noted that these red colored monolayer PET bottles showed poor oxygen scavenging performance with >1.8 ppm oxygen ingress in the first 4 weeks due to an induction period for oxygen scavenging.

### Example 4

A dry blend of “2512 PET resin” with 1 wt % of “OS-A” additive, 0.4 wt % Safflower oil (from a local grocery store) and 0.6 wt % Penn Color Red 66R8980 was made. This dry blend was directly injection molded into preforms which were subsequently blown into monolayer 20 oz. ketchup bottles using the 2 step process described before. These red colored monolayer bottles were tested for oxygen scavenging performance. The oxygen ingress data for this example is shown in FIG. 4. It may be noted that these red colored monolayer PET bottles showed improved oxygen scavenging performance relative to Control Example 4.

### Example 5

This example is identical to Example 4 except 0.4 wt % Corn oil was used in place of Safflower oil. Again from FIG. 4, it may be noted that the red colored monolayer PET bottles from this example also showed improved oxygen scavenging performance relative to Control Example 4.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and scope of the invention, and all such variations are intended to be included within the scope of the following claims.

1. A composition comprising:
a polyester base polymer that is substantially free of antimony and substantially free of phosphorous;
an oxidizable polyether-based additive;
a transition metal catalyst;
a colorant; and
a polyunsaturated fatty ester oil with the formula R—[OCOCₓH_{y}], wherein,
R is an alkyl, a alkylene, a trivalent alkane group, or a glyceryl moiety;
n ranges from 1 to 3;
x ranges from 16 to 20; and
y ranges from 27 to 35.

2. The composition of claim 1, wherein the polyester base polymer contains less than 100 ppm each of antimony and phosphorous.

3. The composition of claim 1, wherein the polyester base polymer contains less than 10 ppm each of antimony and phosphorous.

4. The composition of claim 1, wherein the polyester base polymer comprises polyethylene terephthalate.

5. The composition of claim 1, wherein the oxidizable polyether-based additive has the formula X—[R—O]ₙ—R′—Y, wherein
R is a substituted or unsubstituted alkylene chain having from 2 to 10 carbon atoms;
n ranges from 4 to 100;
X and Y are selected from the group consisting of H, OH, —OCOR₁, —OCOAr₁, —OR₁, and —OAr₁, wherein R₁ is an alkyl group having from 2 to 18 carbon atoms and Ar₁ is an aryl group; and
R′ may be the same as R or selected from the group consisting of
—[COR₂COOR₃O]ₚ— and —[COAr₂COOR₃O]ₚ— wherein Ar₂ is a phenylene or naphthylene group, R₂ and R₃ are C₂ to C₁₈ alkylene groups, and p ranges from 10 to 100.

6. The composition of claim 1, wherein the oxidizable polyether-based additive is selected from the group consisting of polyether diols, ester capped derivatives of polyether diols, polyether-polyester block copolymers, and ether end-capped ether end-capped derivatives of polyether diols.

7. The composition of claim 6, wherein the oxidizable polyether-based additive comprises polytetramethylene ether glyol, PTMEG-b-PET copolymer, or polytetramethylene ether glycol dimethyl ether.

8. The composition of claim 1, wherein the transition metal catalyst comprises cobalt, a carboxylate salt, or cobalt neodecanoate.

9. The composition of claim 1, wherein the polyunsaturated fatty ester oil additive is selected from the group consisting of corn oil, cottonseed oil, flaxseed/linseed oil, grapeseed oil, hemp oil, pumpkin seed oil, safflower oil, soybean oil, sunflower oil, and walnut oil.

10. The composition of claim 1, wherein R is a glyceryl moiety; n=3; x=18 and y=33-35.

11. A wall for a package comprising one layer, the one layer comprising a composition, the composition comprising:
a polyester base polymer that is substantially free of antimony and substantially free of phosphorous;
an oxidizable polyether-based additive;
a transition metal catalyst;
a colorant; and
a polyunsaturated fatty ester oil additive with the formula R—[OCOCₓH_{y}]ₙ wherein,
R is an alkyl, a alkylene, a trivalent alkane group, or a glyceryl moiety;
n ranges from 1 to 3;
x ranges from 16 to 20; and
y ranges from 27 to 35.

12. The wall of claim 11, wherein the polyester base polymer contains less than 10 ppm each of antimony and phosphorous.

13. The wall of claim 11, wherein the polyester base polymer comprises polyethylene terephthalate.

14. The wall of claim 11, wherein the oxidizable polyether-based additive has the formula X—[R—O]ₙ—R′—Y, wherein
R is a substituted or unsubstituted alkylene chain having from 2 to 10 carbon atoms;
n ranges from 4 to 100;
X and Y are selected from the group consisting of H, OH, —OCOR₁, —OCOAr₁, —OR₁, and —OAr₁, wherein R₁ is an alkyl group having from 2 to 18 carbon atoms and Ar₁ is an aryl group; and
R′ may be the same as R or selected from the group consisting of
—[COR₂COOR₃O]ₚ— and —[COAr₂COOR₃O]ₚ— wherein Ar₂ is a phenylene or naphthylene group, R₂ and R₃ are C₂ to C₁₈ alkylene groups, and p ranges from 10 to 100.

15. The wall of claim 11, wherein the oxidizable polyether-based additive is selected from the group consisting of polyether diols, ester capped derivatives of polyether diols, polyether-polyester block copolymers, and ether end-capped ether end-capped derivatives of polyether diols.

16. The wall of claim 15, wherein the oxidizable polyether-based additive is selected from the group consisting of polyether diols, ester capped derivatives of polyether diols, polyether-polyester block copolymers, and ether end-capped ether end-capped derivatives of polyether diols.

17. The wall of claim 11, wherein the oxidizable polyether-based additive comprises polytetramethylene ether glyol, PTMEG-b-PET copolymer, or polytetramethylene ether glycol dimethyl ether.

18. The wall of claim 11, wherein the transition metal catalyst comprises cobalt, a carboxylate salt, or cobalt neodecanoate.

19. The wall of claim 11, wherein the polyunsaturated fatty ester oil additive is selected from the group consisting of corn oil, cottonseed oil, flaxseed/linseed oil, grapeseed oil, hemp oil, pumpkin seed oil, safflower oil, soybean oil, sunflower oil, and walnut oil.

20. The wall of claim 11, wherein R is a glyceryl moiety; n=3; x=18 and y=33-35.
